SEQUENCE LISTING (21 sequences)

SEQ ID NO: 1 - DNA, 270 bp, Apostichopus japonicus
  source 1..270: mol_type = genomic DNA
tggaaccctccacagctctaagagtaaatatgaacattccatttcctaacaaggtacagaaagcatgtacatttttatgaaattgttatttgcatactaaatccgaggaaagttcaaacttataatatgggttcaacctatacagttcaacttccttggtcccatattacagtattggaatggtcttgtcagttttatctcatttataatgcagtcattgtctgtagtagtgtaaatgaatgttgctaaagagctgatatctcatgctgg

SEQ ID NO: 2 - DNA, 24 bp, synthetic construct
  source 1..24: mol_type = other DNA; note = a forward primer for detecting the molecular marker C77185, for sex identification of Apostichopus japonicus
tccagcatgagatatcagctcttt

SEQ ID NO: 3 - DNA, 20 bp, synthetic construct
  source 1..20: mol_type = other DNA; note = a reverse primer for detecting the molecular marker C77185, for sex identification of Apostichopus japonicus
tggaaccctcagcagctcta

SEQ ID NO: 4 - DNA, 25 bp, synthetic construct
  source 1..25: mol_type = other DNA
cgcctctggtatgatgtggttaagc

SEQ ID NO: 5 - DNA, 25 bp, synthetic construct
  source 1..25: mol_type = other DNA
ctgctctggagtcttgctatgtgtc

SEQ ID NO: 6 - DNA, 25 bp, synthetic construct
  source 1..25: mol_type = other DNA
ttggcttgacaaacaacagttagcg

SEQ ID NO: 7 - DNA, 24 bp, synthetic construct
  source 1..24: mol_type = other DNA
ccaactgcaagagggagtatcagc

SEQ ID NO: 8 - DNA, 20 bp, synthetic construct
  source 1..20: mol_type = other DNA
ccacgaacacatgcacacac

SEQ ID NO: 9 - DNA, 22 bp, synthetic construct
  source 1..22: mol_type = other DNA
cggaaatgggagtgctaacttc

SEQ ID NO: 10 - DNA, 23 bp, synthetic construct
  source 1..23: mol_type = other DNA
agtgacggttaagtagtcttgct

SEQ ID NO: 11 - DNA, 20 bp, synthetic construct
  source 1..20: mol_type = other DNA
catggtgatgccgcttttgc

SEQ ID NO: 12 - DNA, 25 bp, synthetic construct
  source 1..25: mol_type = other DNA
agtctgacatgcatccttcttgacc

SEQ ID NO: 13 - DNA, 25 bp, synthetic construct
  source 1..25: mol_type = other DNA
ggaatggtgacgcaaattaggcttc

SEQ ID NO: 14 - DNA, 21 bp, synthetic construct
  source 1..21: mol_type = other DNA
gacctgtggcctttgaacttg

SEQ ID NO: 15 - DNA, 24 bp, synthetic construct
  source 1..24: mol_type = other DNA
catgtaggagtgacgatgatctga

SEQ ID NO: 16 - DNA, 23 bp, synthetic construct
  source 1..23: mol_type = other DNA
gccactcccttcattcgacactg

SEQ ID NO: 17 - DNA, 25 bp, synthetic construct
  source 1..25: mol_type = other DNA
tggaagctgctactaatgactgtgc

SEQ ID NO: 18 - DNA, 22 bp, synthetic construct
  source 1..22: mol_type = other DNA
acacagttgacctttgtgctct

SEQ ID NO: 19 - DNA, 23 bp, synthetic construct
  source 1..23: mol_type = other DNA
acatgtagggatggaaagagggt

SEQ ID NO: 20 - DNA, 25 bp, synthetic construct
  source 1..25: mol_type = other DNA
gtgctggcacagatggaaaacaatg

SEQ ID NO: 21 - DNA, 25 bp, synthetic construct
  source 1..25: mol_type = other DNA
tgtccaggattaaccatcccgaatg

(19) United States
(12) Patent Application Publication
(10) Pub. No.: US 2025/0101515 A1
(43) Pub. Date: Mar. 27, 2025

(54) MOLECULAR MARKER FOR SEX IDENTIFICATION OF APOSTICHOPUS JAPONICUS AND USE THEREOF

(71) Applicant: Institute of Oceanology, Chinese Academy of Sciences, Qingdao City, Shandong Province (CN)

(72) Inventors: Lina SUN, Qingdao City, Shandong Province (CN); Chunxi JIANG, Qingdao City, Shandong Province (CN); Hongyuan ZHAO, Qingdao City, Shandong Province (CN); Shilin LIU, Qingdao City, Shandong Province (CN); Wei CUI, Qingdao City, Shandong Province (CN); Shuai XU, Qingdao City, Shandong Province (CN); Fang SU, Qingdao City, Shandong Province (CN); Hongsheng YANG, Qingdao City, Shandong Province (CN)

(21) Appl. No.: 18/895,886

(22) Filed: Sep. 25, 2024

(30) Foreign Application Priority Data
Sep. 27, 2023 (CN) 202311254175.0

(51) Int. Cl. C12Q 1/6879; C12Q 1/6888

(52) U.S. Cl. CPC C12Q 1/6879; C12Q 1/6888; C12Q 2600/124; C12Q 2600/156

(57) ABSTRACT

The application belongs to the technical field of sex identification of Apostichopus japonicus, and particularly relates to a molecular marker for sex identification of Apostichopus japonicus and use thereof. The molecular marker, C77185, has a nucleotide sequence set forth in SEQ ID NO: 1. The molecular marker for sex identification of Apostichopus japonicus provided by this application may realize accurate sex identification of Apostichopus japonicus from different geographical populations, which is characteristic of practicability, accuracy, high efficiency and the like, and may be used in the actual production process. The molecular marker may be further used for in vivo sex identification in related research of the Apostichopus japonicus, thereby conducting experimental research on a specific sex.

### CROSS REFERENCE TO RELATED APPLICATION

This patent application claims the benefit and priority of Chinese Patent Application No. 202311254175.0 filed with the China National Intellectual Property Administration on Sep. 27, 2023, entitled “MOLECULAR MARKER FOR SEX IDENTIFICATION OF APOSTICHOPUS JAPONICUS AND USE THEREOF” the disclosure of which is incorporated by reference herein in its entirety as part of the present application.

### REFERENCE TO SEQUENCE LISTING

A computer readable XML file entitled “GWP20240604467-SEQUENCE LISTING”, that was created on Jul. 24, 2024, with a file size of about 20,479 bytes, containing the sequence listing for this application, has been filed with this application, and is hereby incorporated by reference in its entirety.

### TECHNICAL FIELD

The present disclosure belongs to the technical field of sex identification of Apostichopus japonicus, and particularly relates to a molecular marker for sex identification of Apostichopus japonicus and use thereof.

### BACKGROUND

Apostichopus japonicus, also known as sea cucumber, belongs to the genus Apostichopus, family Stichopodae, order Synalactida, class Holothuroidea, phylum Echinodermata. It naturally inhabits the northern western Pacific Ocean, including the Far East coast of Russia, the coasts of Japan and South Korea, and the Yellow Sea and the Bohai Sea areas of China. It is the most common marine invertebrate. Apostichopus japonicus is rich in bioactive substances such as polysaccharides and saponins, which is one of the species with the largest output per unit in mariculture in China. According to the statistics of “China Fishery Statistical Yearbook 2022”, in 2021, the aquaculture area of Apostichopus japonicus was 3.71 million mu (1 mu is approximately 0.0667 hectares), the output was 222,700 tons, and the output value of the primary, secondary and tertiary industries exceeded 100 billion yuan (RMB). However, the sex identification of Apostichopus japonicus has become one of the key issues restricting their industrial development. Apostichopus japonicus does not have obvious sexual dimorphism, and it is difficult to distinguish male and female through their phenotype before releasing sperm and eggs during the breeding period. Therefore, it is highly susceptible to a decrease in fertilization and hatching rates due to an imbalanced male to female ratio, thereby increasing mariculture costs. Sex control technology is indispensable in mariculture. Elucidating the molecular mechanism of sex determination and differentiation in sea cucumber is a hot issue in echinoderm genetics, breeding science and evolutionary biology. DNA molecular marker is an important tool for sex identification, which is not limited by development period and tissue site, and can realize in vivo identification. It is practical, accurate and efficient. In order to improve the mariculture efficiency and further carry out the genetic breeding and sex research of Apostichopus japonicus, it is urgent to establish a reliable method for sex identification of Apostichopus japonicus based on DNA molecular markers.

At present, only one DNA molecular marker and identification method for the in vivo identification of Apostichopus japonicus has been published, and there are some shortcomings, such as low accuracy, high false positive rate (females are prone to amplifying dark bands), and the results being possibly affected by the distribution of geographical population. It is urgent to develop an accurate and efficient molecular marker and method suitable for the identification of male and female Apostichopus japonicus distributed across different geographical populations.

### SUMMARY

The present disclosure effectively solves the above problems, and provides a rapid, accurate and reliable molecular marker for sex identification of Apostichopus japonicus and use thereof.

To achieve the above objective, the present disclosure adopts the following technical solutions:

A molecular marker for sex identification of Apostichopus japonicus is provided. The molecular marker, C77185, has a nucleotide sequence set forth in SEQ ID NO: 1.

Use of the molecular marker for sex identification of Apostichopus japonicus, and use of the molecular marker in sex identification of Apostichopus japonicus are provided.

Apostichopus japonicus having the molecular marker C77185 is male.

A primer pair for detecting the molecular marker for sex identification of Apostichopus japonicus is provided. The primer pair has nucleotide sequences as follows:

C77185F:
(SEQ ID NO: 2)
TCCAGCATGAGATATCAGCTCTTT;
and
C77185R:
(SEQ ID NO: 3)
TGGAACCCTCAGCAGCTCTA.

Use of the primer pair in sex identification of Apostichopus japonicas is provided.

A kit including the primer pair for amplification of a molecular marker for sex identification of Apostichopus japonicus is provided.

Use of the kit in sex identification of Apostichopus japonicus is provided.

A method for sex identification of Apostichopus japonicus is provided, including the following steps:
step S1, extracting genomic DNA from the Apostichopus japonicus;
step S2, conducting PCR amplification by using the genomic DNA as a template, where primers used for the PCR amplification are sequences of the above primer pair; and
step S3, conducting agarose gel electrophoresis on a PCR product, wherein the PCR product having a specifically amplified band indicates a male, otherwise, the PCR product indicates a female.

The template of the genomic DNA of the Apostichopus japonicus has a concentration in a range of 1-10 ng/μL. Specifically: Apostichopus japonicus muscle or tube feet are collected for genomic DNA extraction as a genomic DNA template of the Apostichopus japonicus.

Reaction conditions for the PCR amplification are as follows: pre-denaturation at 94° C. for 5 min; followed by 35 cycles of denaturation at 94° C. for 30 s, annealing at 68° C. for 30 s, and extension at 72° C. for 30 s; holding at 72° C. for 10 min, and storage at 4° C.

Upstream and downstream primers for a system of the PCR amplification have nucleotide sequences as set forth in SEQ ID NOs: 2 and 3; 1 μL template (template concentration is 1-10 ng/μL), 0.5 μL upstream primer (10 pmole/μL), 0.5 μL downstream primer (10 pmole/μL), 5.5 μL DNase/RNase-free water, and 7.5 μL Taq DNA Polymerase (TaKaRa Ex Taq™ Version 2.0 plus dye), totalling 15 μL.

The PCR product is detected by electrophoresis, and the electrophoresis lasts for 25 min. If a specific band with a length of 270 bp is amplified, an individual is male; otherwise, the individual is female.

The present disclosure has the following advantages:

The present disclosure establishes a rapid and accurate sex identification method suitable for Apostichopus japonicus in various geographical populations. Sex identification can be realized only by extracting genomic DNA from Apostichopus japonicus followed by conventional PCR amplification and electrophoresis, which has an accuracy of 100%.

### BRIEF DESCRIPTION OF THE DRAWINGS

FIG. 1 is an electrophoresis diagram of genetic sex identification result of Apostichopus japonicus using a molecular marker provided by the Example of the present disclosure; wherein, 6 male and 6 female individual samples each with confirmed sex (lanes 1 to 6) are Apostichopus japonicus individuals from Chinese populations of Rizhao City, Shandong Province; Lingshan Island, Shandong Province; Lvshun, Dalian City, Liaoning Province; Laizhou City, Shandong Province; Liugong Island, Shandong Province and Danzi Island, Shandong Province, respectively; and

FIG. 2 is an electrophoresis diagram of genetic sex identification result of Apostichopus japonicus using a molecular marker provided by the Example of the present disclosure; wherein, 12 male and 12 female individual samples each with confirmed sex (lanes 1 to 12) are Apostichopus japonicus individuals from populations in Rizhao, Rizhao, Liugong Island, Liugong Island, Lingshan Island, Lingshan Island, Danzi Island, Danzi Island, Laizhou, Laizhou, Lvshun, and Lvshun, respectively. Samples 1 to 12 (for both males and females) in FIG. 2 and samples 1 to 6 (for both males and females) in FIG. 1 are different individuals, that is, genetic sex of 36 samples with confirmed sex in six geographical populations are accurately verified in total.

### DETAILED DESCRIPTION OF THE EMBODIMENTS

The specific embodiments of the present disclosure will be described below with reference to the Examples. It should be understood that the specific embodiments described herein are only intended to illustrate and explain the present disclosure and are not intended to limit the present disclosure.

In the present disclosure, the 5× sequencing data of all 29 male individual samples and all 33 female individual samples obtained by high-throughput sequencing were pooled. After bioinformatics analysis and BWA alignment, assembly, specific fragment-based screening, filtration, mapping analysis, candidate sequences with significant differences between male and female pools were obtained. Primers were designed according to the candidate sequences, and the genomic DNA from six female and six male Apostichopus japonicus from six geographical populations of Rizhao, Lingshan Island, Lvshun, Laizhou, Liugong Island and Danzi Island were used as templates for PCR amplification, respectively. A DNA molecular marker C77185 that could be amplified and showed a specific band by electrophoresis in male samples and could not be amplified in female samples was obtained. Subsequently, the sample size was expanded, and the primer pair of the molecular marker C77185 was used for PCR amplification. It was verified that the genetic sex was completely consistent with the physiological sex, with an accuracy rate of 100%.

The molecular marker for sex identification of Apostichopus japonicus provided by the present disclosure may realize accurate sex identification of Apostichopus japonicus from different geographical populations, is characteristic of practicability, accuracy, high efficiency and the like, and may be used in the actual production process. For example, distinguishing the sex of the Apostichopus japonicus before artificial induction of ovulation, thereby effectively controlling the sperm-egg ratio, increasing the fertilization rate, and saving the mariculture cost. The molecular marker may be further used for in vivo sex identification in related research of the Apostichopus japonicus, for example, extracting DNA of the Apostichopus japonicus from tissues such as tube feet for early male and female differentiation, thereby conducting experimental research on a specific sex.

### Example 1

### 1. Sampling and DNA Extraction

The muscle tissues from 33 female Apostichopus japonicus individuals and 29 male Apostichopus japonicus individuals from six geographical populations of Rizhao, Liugong Island, Lingshan Island, Danzi Island, Laizhou and Lvshun were collected, respectively. The muscle tissues of Apostichopus japonicus were frozen in liquid nitrogen and stored in a refrigerator at −80° C. DNA extracts were prepared by rapid nucleic acid lysis method.

2. Screening and Analysis of Sex-related Fragments of Apostichopus japonicus

The above samples were re-sequenced by Novaseq technology, and the 5× data of all male and female samples were merged into a pool. Subsequently, the male pooled samples were aligned to the original female reference genome by BWA, with a mapping rate of 93.88%. The unaligned reads were assembled by SOAPdenovo software, and the K-mer was set to 31 to assemble a genome XY-1. Using the above assembled genome (XY-1) as a reference genome, the female pooling data were aligned by BWA to screen a female assembled fragment without male data alignment (XY.fa). The above fragments were aligned to the female reference genome on blast using blastn of ncbi-blast-2.2.29+, respectively, and the results were screened according to the principle that the length of the alignment exceeded 60% of the length of the contig. A total of 1731 contigs were aligned. The above 1731 contigs were removed from XY.fa, and the remaining contigs (Contigs_1.fa) were retained. Using XY-1 as a reference genome, one male sample was randomly selected and aligned to the reference genome, and contigs with coverage greater than 20× (Contigs_2.fa) were counted. Contigs_1.fa and Contigs_2.fa were intersected, and 1981 contigs were further obtained. Then, with one female genome and one male genome as a reference, respectively, the above contigs were screened by blastn alignment method. The contigs on the female genome and the unaligned contigs in the male genome were filtered out, and finally 36 contigs were obtained.

3. Verification of Molecular Marker for Genetic Sex Identification of Apostichopus japonicus

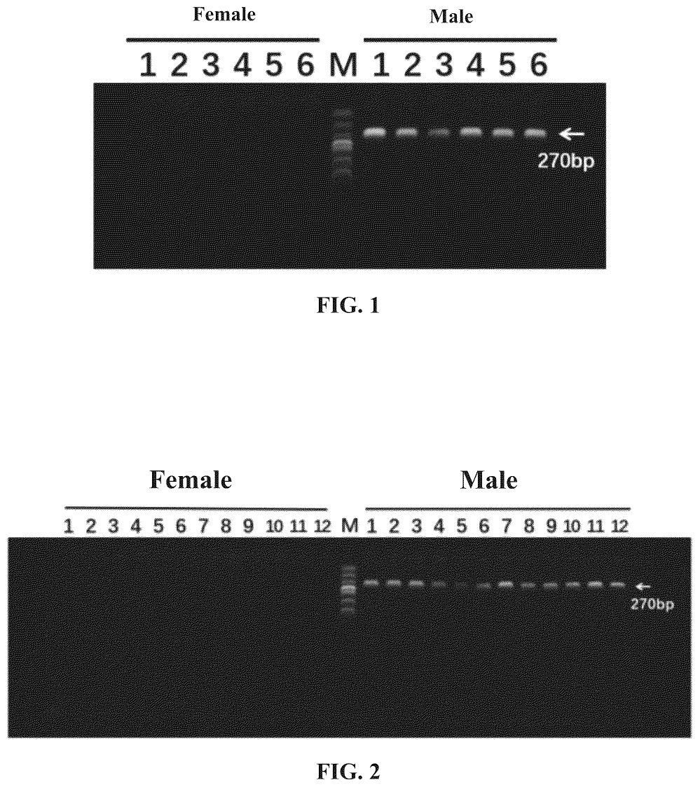

Primers for the above 36 contigs were designed and synthesized by Sangon Biotech. Among them, 26 contigs were too short or high in the AT value to design primers. Finally, 10 primer pairs were synthesized. The genomic DNA of six female and six male Apostichopus japonicus individuals from six geographical populations were extracted and used as templates, together with the above 10 primer pairs for PCR amplification, respectively, and a DNA molecular marker C77185 that could be amplified and showed a specific band by electrophoresis in male samples and could not be amplified in female samples was screened, with a length of 270 bp (see FIG. 1).

TABLE 1
The amplification results using the 10 pairs of primers in male and female
samples

| Contig | Primer pair | Amplification result |
|---|---|---|
| C153960 | F: CGCCTCTGGTATGATGTGGTTAAGC | Males have a bright band; some |
| | (SEQ ID NO: 4) | females have a dark band |
| | R: CTGCTCTGGAGTCTTGCTATGTGTC | |
| | (SEQ ID NO: 5) | |
| C142561 | F: TTGGCTTGACAAACAACAGTTAGCG | Males have a bright band; some |
| | (SEQ ID NO: 6) | females have dark bands |
| | R: CCAACTGCAAGAGGGAGTATCAGC | |
| | (SEQ ID NO: 7) | |
| C103296 | F: CCACGAACACATGCACACAC (SEQ ID | Males have a bright band; some |
| | NO: 8) | females have a plurality of bright |
| | R: CGGAAATGGGAGTGCTAACTTC (SEQ | bands |
| | ID NO: 9) | |
| C82913 | F: AGTGACGGTTAAGTAGTCTTGCT (SEQ | Males have a bright band; some |
| | ID NO: 10) | females have a dark band |
| | R: CATGGTGATGCCGCTTTTGC (SEQ ID | |
| | NO: 11) | |
| C77185 | F: TCCAGCATGAGATATCAGCTCTTT | Males have a bright band; |
| | (SEQ ID NO: 2) | females have no band |
| | R: TGGAACCCTCAGCAGCTCTA (SEQ ID | |
| | NO: 3) | |
| C73206 | F: AGTCTGACATGCATCCTTCTTGACC | Males have a bright band; some |
| | (SEQ ID NO: 12) | females have a dark band |
| | R: GGAATGGTGACGCAAATTAGGCTTC | |
| | (SEQ ID NO: 13) | |
| C56312 | F: GACCTGTGGCCTTTGAACTTG (SEQ | Males have a bright band; some |
| | ID NO: 14) | females have a dark band |
| | R: CATGTAGGAGTGACGATGATCTGA | |
| | (SEQ ID NO: 15) | |
| C53333 | F: GCCACTCCCTTCATTCGACACTG (SEQ | Males have a bright band; some |
| | ID NO: 16) | females have a dark band |
| | R: TGGAAGCTGCTACTAATGACTGTGC | |
| | (SEQ ID NO: 17) | |
| C42834 | F: ACACAGTTGACCTTTGTGCTCT (SEQ | Males have a bright band; some |
| | ID NO: 18) | females have a dark band |
| | R: ACATGTAGGGATGGAAAGAGGGT | |
| | (SEQ ID NO: 19) | |
| C14837 | F: GTGCTGGCACAGATGGAAAACAATG | Males have a bright band; some |
| | (SEQ ID NO: 20) | females have a dark band |
| | R: TGTCCAGGATTAACCATCCCGAATG | |
| | (SEQ ID NO: 21) | |

### Example 2

### 1. Sampling and DNA Extraction

The muscle tissues from 12 male individuals and 12 female individuals of Apostichopus japonicus from six geographical populations were collected, and the genomic DNA of the samples was extracted by rapid nucleic acid lysis method.

### 2. Amplification Conditions of PCR

PCR amplification was conducted with the resulting DNA as a template. The nucleotide sequences of the upstream and downstream primers for the PCR are set forth in SEQ ID NOs: 2 and 3. The PCR system includes 1 μL template (template concentration is 1-10 ng/μL), 0.5 μL upstream primer (10 pmole/μL), 0.5 μL downstream primer (10 pmole/μL), 5.5 μL DNase/RNase-free water, and 7.5 μL Taq DNA Polymerase (TaKaRa Ex Taq™ Version 2.0 plus dye), with a total system of 15 μL. The PCR program is: pre-denaturation at 94° C. for 5 min; followed by 35 cycles of denaturation at 94° C. for 30 s, annealing at 68° C. for 30 s, and extension at 72° C. for 30 s; holding at 72° C. for 10 min, and storage of the PCR product at 4° C.

3. Method for Genetic Sex Identification of Apostichopus japonicus

The PCR product was detected by electrophoresis, and the electrophoresis lasted for 25 min. Those with a specific band of 270 bp in length were male individuals; otherwise, they were female individuals (see FIG. 2). As shown in FIG. 2, the DNA molecular marker C77185 which amplified a specific band in male samples and did not amplified bands in female samples has a length of 270 bp. It is verified that the genetic and physiological sexes of these 24 samples were completely consistent, with an accuracy rate of 100%. And Samples 1 to 12 (for both males and females) in FIG. 2 and samples 1 to 6 (for both males and females) in FIG. 1 were different individuals, that is, genetic sexes of 36 male and female samples in six geographical populations were accurately verified in total.

What is claimed is:

1. A molecular marker C77185 for sex identification of Apostichopus japonicus, wherein the molecular marker has a nucleotide sequence set forth in SEQ ID NO: 1.

2. A method for sex identification of Apostichopus japonicus, comprising using the molecular marker C77185 according to claim 1 as a molecular marker.

3. The method according to claim 2, wherein the Apostichopus japonicus having the molecular marker C77185 is male.

4. A primer pair for detecting the molecular marker C77185 for sex identification of Apostichopus japonicus according to claim 1, wherein the primer pair has nucleotide sequences as follows:
C77185F:
TCCAGCATGAGATATCAGCTCTTT;
and
C77185R:
TGGAACCCTCAGCAGCTCTA.

5. A method for sex identification of Apostichopus japonicus, comprising using the primer pair according to claim 4 as primers for amplification.

6. A kit for amplifying a molecular marker for sex identification of Apostichopus japonicus, comprising the primer pair according to claim 4.

7. A method for sex identification of Apostichopus japonicus, comprising using the kit according to claim 6.

8. A method for sex identification of Apostichopus japonicus, comprising the following steps:
step S1, extracting genomic DNA from the Apostichopus japonicus;
step S2, conducting PCR amplification by using the genomic DNA as a template, wherein primers used for the PCR amplification are sequences of the primer pair according to claim 4; and
step S3, conducting agarose gel electrophoresis on a PCR product, wherein the PCR product having a specifically amplified band indicates a male, otherwise, the PCR product indicates a female.

9. The method for sex identification of Apostichopus japonicus according to claim 8, wherein the template of the genomic DNA of the Apostichopus japonicus has a concentration in a range of 1-10 ng/μL.

10. The method for sex identification of Apostichopus japonicus according to claim 8, wherein reaction conditions for the PCR amplification are as follows: initial denaturation at 94° C. for 5 min, followed by 35 cycles of denaturation at 94° C. for 30 s, annealing at 68° C. for 30 s, and extension at 72° C. for 30 s, holding at 72° C. for 10 min, and storage at 4° C.
